(19) United States
(12) Patent Application Publication
(10) Pub. No.: US 2021/0244580 A1
(43) Pub. Date: Aug. 12, 2021

(54) INCONTINENCE PRODUCT

(71) Applicant: Trunx Wear LLC, Portland, OR (US)

(72) Inventor: Jessica Lubahn, Portland, OR (US)

(21) Appl. No.: 17/173,047

(22) Filed: Feb. 10, 2021

Related U.S. Application Data
(60) Provisional application No. 62/975,067, filed on Feb. 11, 2020.

(51) Int. Cl. A61F 13/537

(52) U.S. Cl. CPC A61F 13/53747; A61F 2013/530299; A61F 13/53704

(57) ABSTRACT

An incontinence product permanently-bonded to an interior garment fabric of an incontinence garment in a position to absorb incontinent leakage. The incontinence product includes a one-way wicking layer configured to receive the incontinent leakage; an outer shell fabric layer adjacent the interior garment fabric; an absorbent fabric layer intermediate the one-way wicking layer and the outer shell fabric layer; and a leakproof hydrophilic layer intermediate the absorbent fabric layer and the outer shell fabric layer. A perimeter portion of the outer shell fabric layer forms a window through which the one-way wicking layer is configured to receive the incontinent leakage. Stitching through the one-way wicking layer, the absorbent fabric layer, the leakproof hydrophilic layer, and a portion of the outer shell fabric layer adjacent the interior garment fabric holds in place relative to one another the one-way wicking layer, the absorbent fabric layer, the leakproof hydrophilic layer, and the portion of the outer shell fabric layer adjacent the interior garment fabric.

### CROSS REFERENCE TO RELATED APPLICATIONS

This application claims priority to U.S. Provisional Patent Application Ser. No. 62/975,067, filed Feb. 11, 2020, the entirety of which is hereby incorporated herein by reference for all purposes.

### BRIEF DESCRIPTION OF THE DRAWINGS

FIG. 1A shows material layers for an incontinence product.

FIG. 1B shows a stack of material layers in an incontinence product.

FIG. 1C-1D show an exemplary folding and stitching pattern for assembling an incontinence product.

FIG. 1E shows a cross section of an incontinence product.

FIG. 1F show an alternate shape for an outermost layer of an incontinence product.

FIG. 1G shows a stack of material layers using the alternate shape for the outermost layer.

FIG. 1H-1I show an alternate exemplary folding and stitching pattern for assembling an incontinence product using the alternate shape for the outermost layer.

FIGS. 2A-2C illustrate usage of an incontinence product by bonding with a garment.

FIG. 3 shows an incontinence product with a garment built around the incontinence product.

FIG. 4 shows another incontinence product with a garment built around the incontinence product.

### DETAILED DESCRIPTION

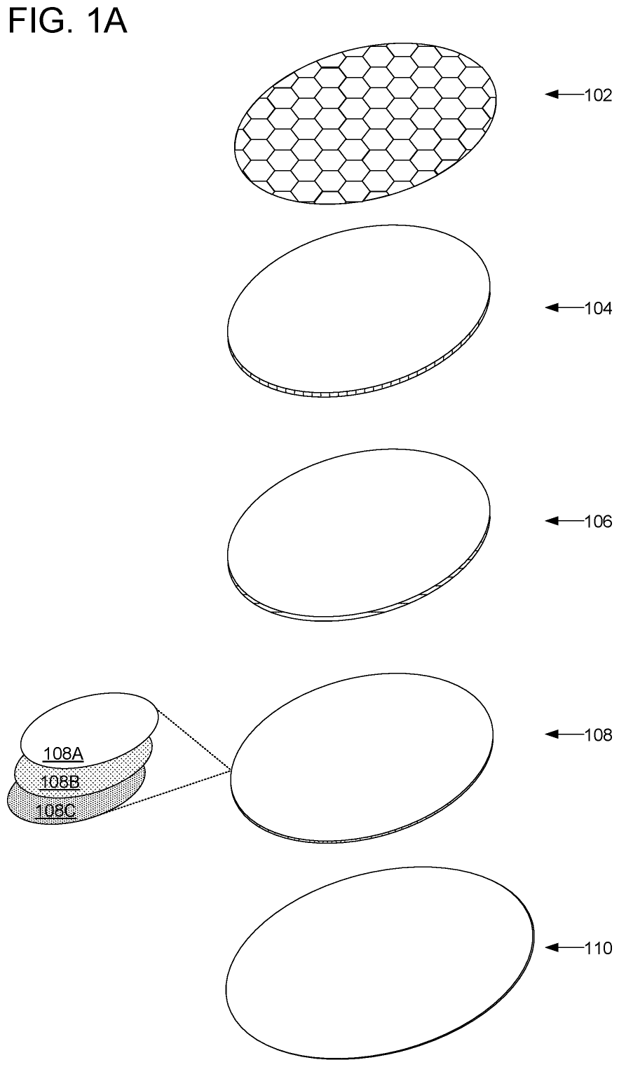

FIG. 1A shows material layers of an incontinence product, including a one-way wicking fabric layer 102, a first absorbent fabric layer 104, an optional second absorbent fabric layer 106, a hydrophilic and leakproof fabric layer 108, and an outer shell fabric layer 110. The layers are cut into an elliptical shape, which is one non-limiting example of a shape for the layers. Any other shape may be used. The outer shell fabric layer 110 has a larger size than the other layers. The outer shell fabric layer 110 may be waterproof.

Any suitable one-way wicking fabric may be used. As one non-limiting example, the one-way wicking fabric layer 102 may be 81% modal, 13% alginate fiber, and 6% spandex. In some examples, two or more one-way wicking fabric layers may be used. When two or more one-way wicking fabric layers are used, secondary one-way wicking fabric layers may be placed immediately adjacent the other one-way wicking fabric layer (e.g., at the top of the stack) or at other positions within the stack of layers.

One or more absorbent fabric layers may be used. Although some examples herein show two absorbent fabric layers (absorbent fabric layer 104 and optional second absorbent fabric layer 106), in some examples, only one absorbent fabric layer may be used (e.g., absorbent fabric layer 104 may be used without any other absorbent fabric layers). When two or more absorbent fabric layers are used, all layers may be the same, or one or more layers may be different than one or more other layers. Any suitable absorbent fabric(s) may be used. As one non-limiting example, either or both of absorbent fabric layer 104 and/or absorbent fabric layer 106 may be 100% polyester. When two or more absorbent fabric layers are used, secondary absorbent fabric layers may be placed immediately adjacent absorbent fabric layer 104 and/or absorbent fabric layer 106, or at other positions within the stack of layers.

Any suitable hydrophilic and/or leakproof materials may be used. As one non-limiting example, hydrophilic and leakproof layer 108 may be 85% polyester, 10% elastane, and 5% polyurethane. In some examples, hydrophilic and leakproof layer 108 may be made of a plurality of sub-layers that are bonded together. For example, as shown in FIG. 1A, hydrophilic and leakproof layer 108 includes a hydrophilic layer 108A, a leakproof film 108B, and a liner fabric layer 108C, with the three layers bonded together. The liner fabric layer 108C may protect the leakproof film.

Any suitable outer shell fabric may be used. As one non-limiting example, the outer shell fabric layer 110 may be 70% nylon and 30% spandex. FIG. 1B shows material layers of an incontinence product cut and stacked for assembly. The fabric layer 102, first absorbent fabric layer 104, second absorbent fabric layer 106, and hydrophilic and leakproof fabric layer 108 are stacked so that the cut sides of the layers are aligned. These layers are aligned atop the outer shell fabric layer 110, which protrudes past the other layers (e.g., due to its larger size). The incontinence product may include other components not shown in FIG. 1B, for example, adhesives (e.g., waterproof bonding tape) and/or other attachments (e.g., thread). For example, waterproof bonding tape may be applied around a perimeter of the stack shown in FIG. 1B.

FIG. 1C shows layers of an incontinence product stacked for assembly, with the outer shell fabric layer 110 folded around edges of the other layers, so that outer shell fabric layer 110 forms a perimeter around the other layers and only the top one-way wicking fabric layer 102 is exposed through a window established by the folded perimeter of the outer shell fabric layer. FIG. 1C is one non-limiting example of a folding pattern for folding the outer shell fabric layer 110 around the other layers, wherein outer shell fabric layer 110 is folded first along edges indicated 110A, and then folded along edges indicated 110B. The outer shell fabric layer 110 may be variously shaped and sized to accommodate any desired folding pattern and final folded shape and configuration. In some embodiments, the outer shell fabric layer will form a ring atop the other layers, thereby effectively sealing the bottom, sides, and top edges of the incontinence product from liquid leakage. In other embodiments, the outer fabric layer is fabricated as a pocket that substantially mimics the folding arrangement described above without requiring folding.

FIG. 1D shows an exemplary completed assembly for an incontinence product 100. The outer shell fabric layer 110 is folded around other layers to form a perimeter so that only the top one-way wicking fabric layer 102 is exposed within the perimeter (e.g., according to the folding pattern in FIG. 1C). Stitching 112 is sewn around the perimeter formed by the folded edges of outer shell fabric layer 110. For example, stitching 112 may protrude through a top folded portion of outer shell fabric layer 110, through one-way wicking fabric layer 102, first absorbent fabric layer 104, second absorbent fabric layer 106, hydrophilic and leakproof fabric layer 108, and a bottom portion of outer shell fabric layer 110. Although not shown in FIG. 1D, the incontinence product 100 assembly may also include other adhesives (e.g., waterproof bonding tape) and/or other components (e.g., additional stitching). Stitching 112 is one non-limiting example of a stitching pattern for joining the layers of the incontinence product 100, suitable for the folding pattern shown in FIGS. 1C-1D. In general, stitching to join the layers may follow any suitable pattern so as to closely join the folded edges of the outer shell fabric layer 110 to the other layers (e.g., to prevent an excessive flap portion of the folded outer shell fabric layer 110 from being freely movable away from the other layers).

FIG. 1E shows a cross section of an incontinence product 100, including a main stack of layers including one-way wicking fabric layer 102, first absorbent fabric layer 104, second absorbent fabric layer 106, and hydrophilic and leakproof fabric layer 108. The layers are stitched together with stitching 112. The stack of layers are bonded around a perimeter with an optional layer of waterproof bonding tape 114. When included, the optional layer of waterproof bonding tape may prevent leakage made possible by the stitching. In other implementations, a sealant optionally may be used to reduce or eliminate leakage through the stitching.

The outer shell fabric layer 110 forms the bottom of the main stack of layers, and is also folded around the perimeter over the top of the other layers to cover waterproof bonding tape 114 and one-way wicking fabric layer 102. Although waterproof bonding tape 114 is shown between outer shell fabric layer 110 and other layers, in some examples, waterproof bonding tape 114 may alternately or additionally be applied to an outside surface of outer shell fabric layer 110.

FIGS. 1F-1I show an alternate assembly for an incontinence product with an alternate non-limiting example of a folding and stitching pattern (e.g., as compared to the incontinence product illustrated in FIGS. 1A-1E). FIG. 1F shows an alternate shape for an outer shell fabric layer 110′. FIG. 1G shows a stack of layers atop the alternative shape for the outer shell fabric layer 110′, including one-way wicking fabric layer 102, first absorbent fabric layer 104, second absorbent fabric layer 106, and hydrophilic and leakproof fabric layer 108. FIG. 1H shows outer shell fabric layer 110′ folded around the stack of layers to form a perimeter so that only the top one-way wicking fabric layer 102 is exposed within the perimeter. The folding pattern shown in FIG. 1H may be obtained by folding outer shell fabric layer 110′ analogously to the folding pattern for outer shell fabric layer 110 as shown in FIG. 1C. FIG. 1I shows an alternate assembled incontinence product 100′, using the alternate shape of outer shell fabric layer 110′ folded around the other layers and sewn with an alternate stitch pattern 112′.

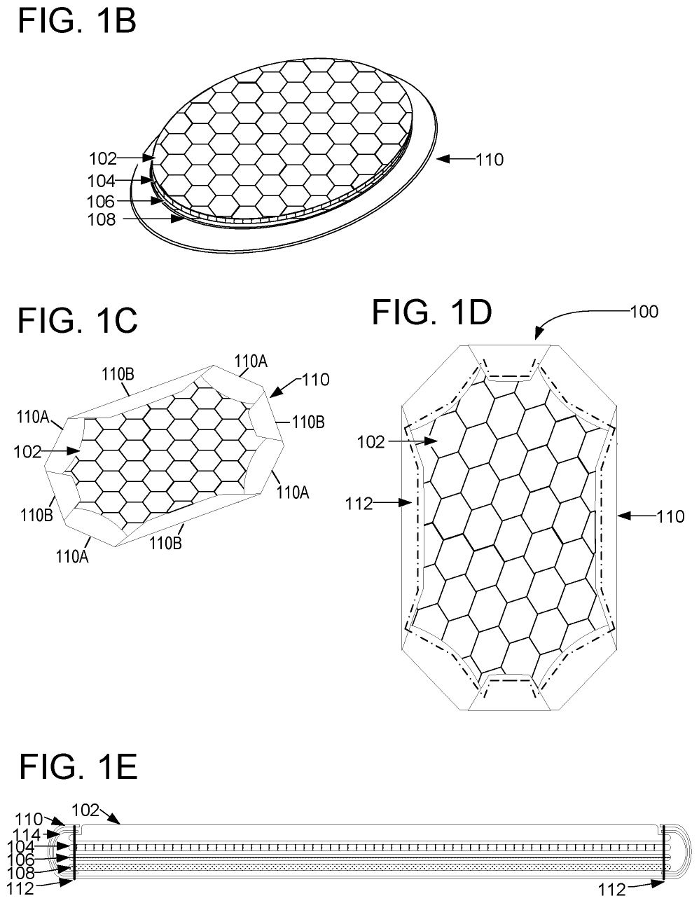

FIG. 2A shows a non-limiting exemplary usage of incontinence product 100, in which incontinence product 100 may be attached to a garment 200. For example, the incontinence product 100 may be bonded to an interior of a garment such as an undergarment (e.g., men's, women's, and/or unisex briefs or boxers, and/or any other product). Incontinence product 100 may be prepared with a stitchless, heat-activated fabric welding system (e.g., BEMIS™ or FRAMIS™ heat-welding systems). For example, an outer surface of outer shell fabric layer 110 may be pre-treated with the heat-activated fabric weld. As shown in FIG. 2B, the incontinence product 100 may be bonded to a fabric surface 200A of the garment (e.g., an inseam or liner of the garment). After bonding, the garment 200 may be washed (e.g., incontinence product 100 does not negatively impact washability and is not damaged by washing) and incontinence product 100 remains absorbent and leak-proof (e.g., bonding to garment 200 does not negatively impact leak-proof, water-proof, and/or absorbency qualities of incontinence product 100). FIG. 2C shows a cross-section of an incontinence product 100 attached to a fabric surface 200A, via a heat-activated fabric weld 116 taping the outer shell fabric layer 110 of incontinence product 100 to the fabric surface 200A.

In some examples, as an alternative to attaching an incontinence product to a pre-fabricated garment, a garment may be built around an incontinence product of arbitrary size/shape. For example, the incontinence product need not conform to a shape of existing garments, and shapes of the incontinence product are not limited to the shapes shown in the present disclosure (e.g., the shapes of incontinence product 100 and 100′ are non-limiting examples of possible shapes for an incontinence product).

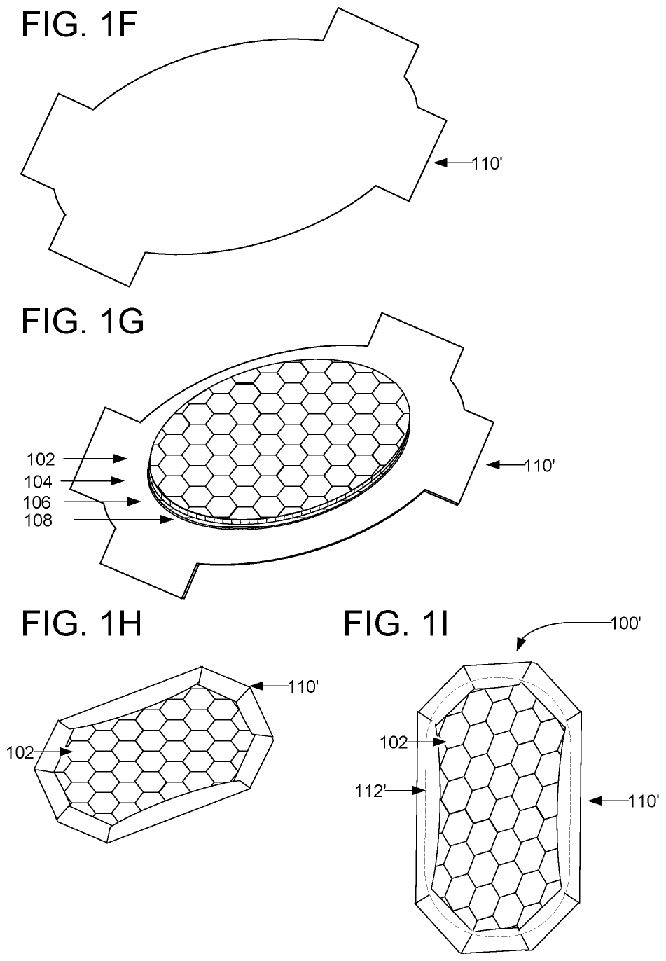

For example, FIG. 3 shows a cross section of a garment including an incontinence product 300. Incontinence product 100 and incontinence product 100′ are non-limiting examples of incontinence product 300, but other incontinence products of arbitrary size and shape with the same or different layering are also envisaged. Incontinence product 300 is attached to two or more fabric surfaces 3000A and 3000A′ via heat-activated fabric weld 316 taping a perimeter of incontinence product 300 to fabric surfaces 350A and 350A′. Fabric surfaces 350A and 350A′ may be any contiguous or non-contiguous fabric portions of any suitable garment). Fabric surfaces 350A and 350A′ may be the same or different fabric(s) from outer shell fabric layer 310 of incontinence product 300. For example, outer shell fabric layer 310 and fabric surfaces 350A and 350A′ may both be water-proof, leak-proof fabric(s); or in some examples fabric layer 310 may be a water-proof, leak-proof fabric while fabric surfaces 350A and 350A′ and/or the remainder of the garment is not water-proof (e.g., cotton).

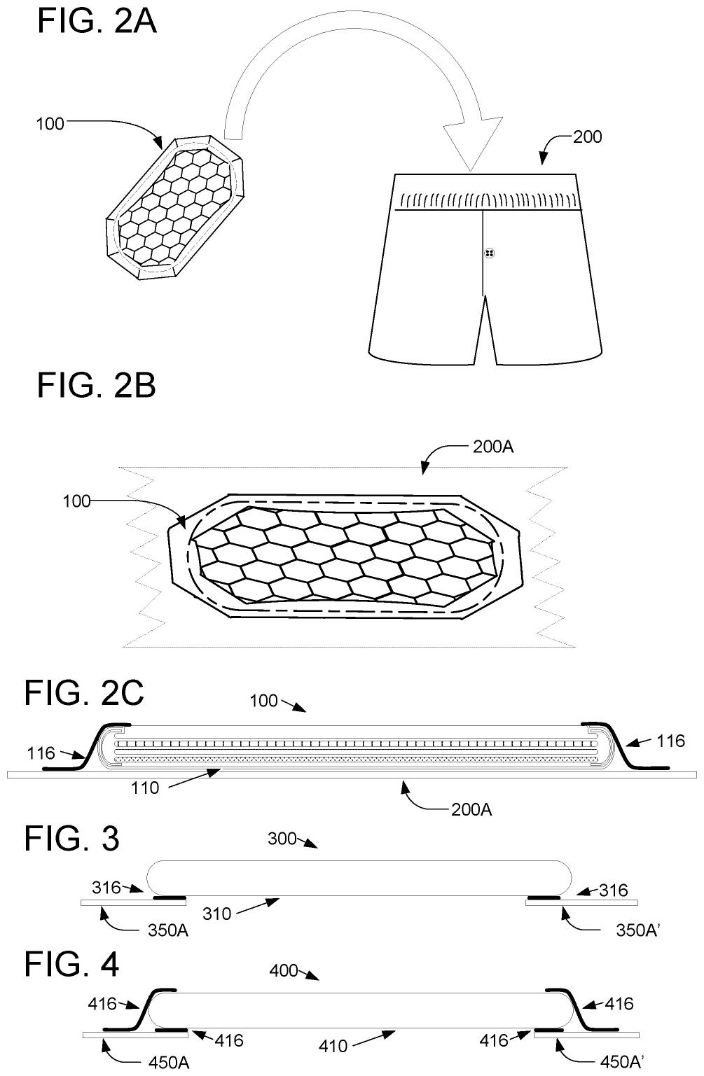

As another alternative construction, FIG. 4 shows an incontinence product 400 (e.g., incontinence product 100 or incontinence product 100′ are non-limiting examples). Incontinence product 400 is attached to fabric surfaces 450A and 450A′ with heat-activated fabric weld 416 attaching multiple portions of outer shell fabric 410 around a perimeter of the incontinence product 400 as shown in cross-section in FIG. 4.

FIGS. 2C, 3, and 4 show an outer shell fabric layer of an incontinence product being adjoined to fabric surfaces with heat-activated fabric weld. Alternately or additionally, any other suitable techniques and/or materials may be used to attach an incontinence product to a garment and/or incorporate incontinence product into a garment (e.g., by building up the garment around the incontinence product). For example, an incontinence product may be attached to fabric surfaces via stitching/seams, adhesives, and/or water-proof/leak-proof tape.

1. An incontinence product permanently-bonded to an interior garment fabric of an incontinence garment in a position to absorb incontinent leakage, the incontinence product comprising:
a one-way wicking layer configured to receive the incontinent leakage;
an outer shell fabric layer adjacent the interior garment fabric;
an absorbent fabric layer intermediate the one-way wicking layer and the outer shell fabric layer;
a leakproof hydrophilic layer intermediate the absorbent fabric layer and the outer shell fabric layer;
wherein a perimeter portion of the outer shell fabric layer forms a window through which the one-way wicking layer is configured to receive the incontinent leakage; and
stitching through the one-way wicking layer, the absorbent fabric layer, the leakproof hydrophilic layer, and a portion of the outer shell fabric layer adjacent the interior garment fabric, thereby holding in place relative to one another the one-way wicking layer, the absorbent fabric layer, the leakproof hydrophilic layer, and the portion of the outer shell fabric layer adjacent the interior garment fabric.

2. The incontinence product of claim 1, wherein the perimeter portion of the outer shell fabric layer is folded around perimeters of the one-way wicking layer, absorbent fabric layer, and leakproof hydrophilic layer, thereby forming the window through which the one-way wicking layer is configured to receive the incontinent leakage.

3. The incontinence product of claim 2, wherein the stitching is through the perimeter portion of the outer shell thereby holding in place relative to one another the perimeter portion of the outer shell, the one-way wicking layer, the absorbent fabric layer, the leakproof hydrophilic layer, and the portion of the outer shell fabric layer adjacent the interior garment fabric.

4. An incontinence garment, comprising:
an interior garment fabric; and
an incontinence product permanently-bonded to the interior garment fabric in a position to absorb incontinent leakage, the incontinence product including:
a one-way wicking layer configured to receive the incontinent leakage;
an outer shell fabric layer adjacent the interior garment fabric;
an absorbent fabric layer intermediate the one-way wicking layer and the outer shell fabric layer;
a leakproof hydrophilic layer intermediate the absorbent fabric layer and the outer shell fabric layer;
wherein a perimeter portion of the outer shell fabric layer forms a window through which the one-way wicking layer is configured to receive the incontinent leakage; and
stitching through the one-way wicking layer, the absorbent fabric layer, the leakproof hydrophilic layer, and a portion of the outer shell fabric layer adjacent the interior garment fabric, thereby holding in place relative to one another the one-way wicking layer, the absorbent fabric layer, the leakproof hydrophilic layer, and the portion of the outer shell fabric layer adjacent the interior garment fabric.

5. The incontinence garment of claim 4, wherein the perimeter portion of the outer shell fabric layer is folded around perimeters of the one-way wicking layer, absorbent fabric layer, and leakproof hydrophilic layer, thereby forming the window through which the one-way wicking layer is configured to receive the incontinent leakage

6. The incontinence garment of claim 5, wherein the stitching is through the perimeter portion of the outer shell thereby holding in place relative to one another the perimeter portion of the outer shell, the one-way wicking layer, the absorbent fabric layer, the leakproof hydrophilic layer, and the portion of the outer shell fabric layer adjacent the interior garment fabric.
